(19) United States
(12) Patent Application Publication
(10) Pub. No.: US 2019/0014806 A1
(43) Pub. Date: Jan. 17, 2019

(54) Composition to Reduce the Negative Effects of Alcohol Consumption

(71) Applicant: Ivan GALANIN, New York, NY (US)

(72) Inventor: Ivan GALANIN, New York, NY (US)

(21) Appl. No.: 15/647,861

(22) Filed: Jul. 12, 2017

(51) Int. Cl. A23L 33/105; A61K 31/185; A23L 33/16; A23L 33/15; A61K 9/00

(52) U.S. Cl. CPC A23L 33/105; A61K 31/185; A61K 9/0053; A23L 33/15; A23L 33/16

(57) ABSTRACT

A composition for reducing blood alcohol concentration (BAC) in a person after alcohol consumption is provided, which contains taurine in a combination with a lipid and/or a natural or synthetic compound. A method of treating a negative effect of alcohol consumption in a person is also provided.

### FIELD OF THE INVENTION

The present invention relates to a composition that can mitigate and reduce the toxic effects of alcohol consumption. More specifically, a composition reduces the blood alcohol level and allows the consumer to experience a faster recovery from the consumption of alcohol.

### BACKGROUND OF THE INVENTION

Excessive alcohol consumption is widespread. There is a tremendous need for products that are safe and effective at alleviating the toxicity of alcohol. Given the myriad of pathologies caused by excessive alcohol consumption, it is not likely that any single agent will be optimally effective.

Taurine has shown some promise at mitigating aspects of alcohol toxicity in animal studies. There is no human clinical evidence that taurine as a standalone treatment can mitigate the toxicity of alcohol consumption in vivo. There is also no human clinical evidence that combining taurine with other ingredients can result in a composition that alleviates alcohol toxicity. For example, energy drinks containing quantities of taurine of 1 gram or more have been evaluated in human clinical studies of alcohol consumption and been shown to have no effect on alcohol metabolism or drinkers' well-being.

Despite the hundreds of botanical and other natural products have been shown to have efficacy in models of alcohol toxicity, there have been no systematic studies to identify which natural products can be combined effectively to treat alcohol toxicity. This is important because many natural products, especially botanical extracts, comprise thousands of ingredients that modulate multiple biological pathways in vivo. Without testing, it is not possible to say which combinations of natural products will have additive or synergistic efficacy and which will counter-act or abrogate single agent activity.

### SUMMARY OF THE INVENTION

Through extensive in vivo testing, this invention identifies surprising combinations of natural products that have additive protection in an animal model of alcohol toxicity. The combinations have also shown efficacy in protecting humans from alcohol exposure. Specifically, they have been shown to reduce the peak blood alcohol attained in subjects consuming alcohol and to increase self-reported quality of life in these subjects.

In one embodiment of the invention, taurine may be combined with a source of lipids to form a composition that has additive protective effects against alcohol toxicity.

In another embodiment of the invention, taurine may be combined with a source of choline to form a composition that has additive protective effects against alcohol toxicity.

In another embodiment of the invention, taurine may be combined with a source of phospholipids to form a composition that has additive protective effects against alcohol toxicity.

In another embodiment of the invention, taurine may be combined with a source of folate to form a composition that has additive protective effects against alcohol toxicity.

This invention used a modified version of the alcohol sedation model disclosed by Sandhu et al to evaluate the effects of acute alcohol exposure in drosophila. For example, in one of the models, for at least two days prior to alcohol exposure, drosophila consumed standard fly food combined with placebo or investigational agents. The drosophila were then moved to empty, foam stopper-covered vials and allowed to acclimate before the foam stoppers were injected with 1 ml 100% ethanol. At different time intervals, it was noted how many drosophila in each vial lost consciousness. After removal of the ethanol-treated foam stoppers, the recovery status of the drosophila was graded at different time intervals: a grade of zero was given to drosophila that were dead or passed out; a grade of 25% was given to drosophila that were twitching but not upright; a grade of 50% was given to drosophila that were upright or walking but not crawling up the side of the vial; and a grade of 100% to drosophila that had crawled on the side of the vial or escaped. The average recovery score was calculated by averaging the recovery scores of individual drosophila. Increasing the extent of alcohol exposure led to greater alcohol toxicity, including substantial death.

Preferred embodiments of this invention were also tested on human subjects to confirm the efficacy of the data retrieved from the drosophila testing. Volunteers were asked to follow a testing protocol that required them to consume a given amount of alcohol over a given period of time with or without food as they preferred. Each volunteer would then measure his or her blood alcohol level (BAC) at various intervals after consuming alcohol using a professional breathalyzer device. Volunteers would conduct the protocol twice: once in the context of consuming the combination of natural products and once without. The efficacy of a preferred embodiment of the invention in the context of consumption of beer, wine, spirits, and mixed drinks by men and women, both fed and fasted, was tested.

Other objects, advantages and novel features of the present invention will become apparent from the following detailed description of one or more preferred embodiments when considered in conjunction with the figures presented. It should be recognized that the one or more examples in the disclosure are non-limiting examples and that the present invention is intended to encompass variations and equivalents of these examples. The disclosure is written for those skilled in the art. Although the disclosure uses terminology and acronyms that may not be familiar to the layperson, those skilled in the art will be familiar with the terminology and acronyms used herein.

### BRIEF DESCRIPTION OF DRAWINGS

FIG. 1 shows the effects of taurine in an animal model of acute alcohol exposure over time.

FIG. 2 shows the effects of Krill oil in combination with taurine in an animal model of acute alcohol exposure over time relative to taurine supplementation alone.

FIG. 3 shows the effects of Castor oil in combination with taurine in an animal model of acute alcohol exposure over time relative to taurine supplementation alone.

FIG. 4 shows the effects of Primrose oil in combination with taurine in an animal model of acute alcohol exposure over time relative to taurine supplementation alone.

FIG. 5 shows the effects of Palm kernel oil in combination with taurine in an animal model of acute alcohol exposure over time relative to taurine supplementation alone.

FIG. 6 shows the effects of Phosal® 53 MCT (soy-based lecithin oil plus medium chain triglycerides) in combination with taurine in an animal model of acute alcohol exposure over time relative to taurine supplementation alone.

FIG. 7 shows the effects of using a composition composed of coconut oil and choline in addition to taurine in an animal model of acute alcohol exposure over time relative to using a composition with Phosal® 53 MCT (soy-based lecithin oil plus medium chain triglycerides) and taurine.

FIG. 8 shows the effects of Curcumin in combination with taurine in an animal model of acute alcohol exposure over time relative to taurine supplementation alone.

FIG. 9 shows the effects of Chicory extract in combination with taurine in an animal model of acute alcohol exposure over time relative to taurine supplementation alone.

FIG. 10 shows the effects of Folic acid in combination with taurine in an animal model of acute alcohol exposure over time relative to taurine supplementation alone.

FIG. 11 shows the effects of adding trace minerals to a composition including taurine in an animal model of acute alcohol exposure over time relative to taurine supplementation alone.

FIG. 12 shows the effects of Baicalein extract in combination with taurine in an animal model of acute alcohol exposure over time relative to taurine supplementation alone.

FIG. 13 shows the effects of adding clove extract to a composition including taurine in an animal model of acute alcohol exposure over time relative to taurine supplementation alone.

FIG. 14 shows the effects of adding watercress extract to a composition including taurine in an animal model of alcohol exposure over time compared to using the same composition without watercress extract.

FIG. 15 shows the effects of adding dihydromyricetin extract to taurine supplementation in an animal model of alcohol exposure over time compared to taurine supplementation alone.

FIG. 16 shows the effects of adding taurine to a commercial liver protection product in an animal model of alcohol exposure over time compared to using the commercial liver protection product without taurine.

FIG. 17 shows the effects of using a commercial hangover product (“Hangover Heaven”) including taurine in an animal model from alcohol exposure over time as opposed to the invention composition including taurine.

FIG. 18 shows the effects of a composition according to an embodiment of the invention on blood alcohol levels (blood alcohol concentration) over time after alcohol consumption in humans from a “binge drinking” experiment.

FIG. 19 shows the effects of a composition according to an embodiment of the invention on blood alcohol levels (blood alcohol concentration) over time after alcohol consumption in humans from a “binge drinking” experiment on an empty stomach.

FIG. 20 shows the effects of a composition according to an embodiment of the invention on blood alcohol levels (blood alcohol concentration) over time after alcohol consumption in humans from a “binge drinking” experiment on an empty stomach.

FIG. 21A shows the effects of a composition according to an embodiment of the invention on blood alcohol levels over minutes after alcohol consumption in a person (subject 1) from a “cocktail” experiment.

FIG. 21B shows the effects of a composition according to an embodiment of the invention on blood alcohol levels over minutes after alcohol consumption in a person (subject 2) from a “cocktail” experiment.

FIG. 22 shows the effects of a composition according to an embodiment of the invention on blood alcohol levels (blood alcohol concentration) over minutes after alcohol consumption in humans from a “wine drinking” experiment.

FIG. 23 shows the effects of a composition according to an embodiment of the invention on blood alcohol levels (blood alcohol concentration) over minutes after alcohol consumption in humans from a “beer drinking” experiment.

### DETAILED DESCRIPTION

Unless defined otherwise, all technical and scientific terms used herein have the same meaning as commonly understood by one of ordinary skill in the art to which this invention belongs.

The present invention relates to a composition that can lower the absorption rate of alcohol, thereby reducing the harmful effects of the alcohol. In one aspect of the invention, a composition for reducing blood alcohol concentration (BAC) in a person after alcohol consumption is provided, which contains taurine in a combination with a lipid and/or a natural or synthetic compound.

In one embodiment of the invention, taurine may be combined with a source of lipids to form a composition that has additive protective effects against alcohol toxicity, in part, by reducing blood alcohol concentration (BAC) in a person. In one embodiment of the invention, the lipid is derived from an oil or oils of plant or animal origin. Examples of lipids include fats, waxes, sterols, fat-soluble vitamins (such as vitamins A, D, E, and K), monoglycerides, diglycerides, triglycerides, phospholipids, and the likes. In a preferred embodiment, the lipids are selected from oils with high phospholipid content (such as krill oil, soy-based lecithin oil plus medium chain triglycerides (Phosal® 53 MCT)) and plant based oils (such as castor oil, primrose oil, palm kernel oil, coconut oil and the likes).

As used herein, “krill” may be any arthropod that belongs to, subclass Malacostraca, class Crustacea, phylum Arthropoda, and includes arthropods that belong to order Euphausiacea, superorder Eucarida, subclass Malacostraca, class Crustacea, phylum Arthropoda, such as Antarctic krill (Euphausia superba), and arthropods that belong to order Mysida, superorder Peracarida, subclass Malacostraca, class Crustacea, phylum Arthropoda, such as mysids caught in the oceans near Japan.

As used herein, “krill oil” refers to the oil obtained from the krill described above.

The krill oil is characterized in that it has high phospholipid content. Phospholipid is known as a major constituent of the cell membrane and refers to a substance that has a hydrophilic phosphate moiety and a hydrophobic fatty acid moiety. Phospholipids are divided into glycerophospholipids and sphingophospholipids by the difference in their skeletal structure. As referred to herein, phospholipids include both types of phospholipids, with glycerophospholipids being preferred. Glycerophospholipids include, but are not limited to, phosphatidyl choline, phosphatidyl ethanolamine, phosphatidyl serine, phosphatidylinositol, phosphatidylglycerol, cardiolipin, and phosphatidic acid, and mixtures of two or more thereof. In the present invention, at least phosphatidyl choline is preferably contained as phospholipids. The phospholipids content in the krill oil is, for example, 5-80% by weight and particularly preferably 30-60% by weight. Alternatively, the krill oil can contain phospholipids in an amount of preferably at least 25% by weight and more preferably at least 35% by weight.

In another embodiment of the invention, taurine may be combined with natural or synthetic compounds to form a composition that has additive protective effects against alcohol toxicity, in part, by reducing blood alcohol concentration (BAC) in a person. Examples of natural or synthetic compounds include herbal extracts and dietary supplements. Examples of herbal extracts and dietary supplements include, but are not limited to, curcumin, chicory extract, baicalein extract, clove extract, and watercress extract. In a preferred embodiment of the invention, taurine may be combined with a source of choline to form a composition that has additive protective effects against alcohol toxicity by reducing the blood alcohol concentration (BAC) in a person. Examples of choline include dietary or supplemental choline. Other examples of a source of choline include choline chloride, choline bitartrate, citicoline (CDP-choline), L-alpha-glycerophosphocholine (Alpha-GPC), lecithin, phosphatidylcholine (PC).

In another preferred embodiment of the invention, taurine may be combined with a source of folate to form a composition that has additive protective effects against alcohol toxicity, in part, by reducing the blood alcohol concentration (BAC) in a person. Examples of folate include dietary or supplemental B vitamins.

In another embodiment of the invention, taurine may be combined with a lipid and a natural or synthetic compound. For example, a composition of the invention can include taurine, coconut oil and choline.

In other embodiments of the invention, taurine may be combined with trace minerals. Trace minerals are elements that are needed by the human body in amounts smaller than 100 mg per day and, typically 1 to 100 mg/day by adults or less than 0.01 percent of total body weight. Examples of trace minerals include copper, chromium, fluoride, iodine, iron, molybdenum, manganese, selenium, and zinc.

Drosophila Testing of Alcohol Toxicity

In the following examples, a modified version of the alcohol sedation model employed by Sandhu et al. (“An Inexpensive, Scalable Behavioral Assay for Measuring Ethanol Sedation Sensitivity and Rapid Tolerance in Drosophila,” J Vis Exp. Author manuscript; available in PMC 2016 Apr. 15) to evaluate the effects of acute alcohol exposure was used. For at least two days prior to alcohol exposure, flies consumed standard fly food combined with placebo or investigational agents. The flies were moved to empty, foam stopper-covered vials and allowed to acclimate before the foam stoppers were injected with 1 ml 100% ethanol. At different time intervals, it was noted how many flies in each vial passed out. After removal of the ethanol-treated foam stoppers, the recovery status of the flies was graded at different time intervals: a grade of zero was given to flies that were dead or passed out, a grade of 25% was given to flies that were twitching but not upright, a grade of 50% was given to flies that were upright or walking but not crawling up the side of the vial and a grade of 100% to flies that had crawled on the side of the vial or escaped. The average recovery score is calculated by averaging the recovery scores of individual flies. Increasing the extent of alcohol exposure led to greater alcohol toxicity, including substantial death.

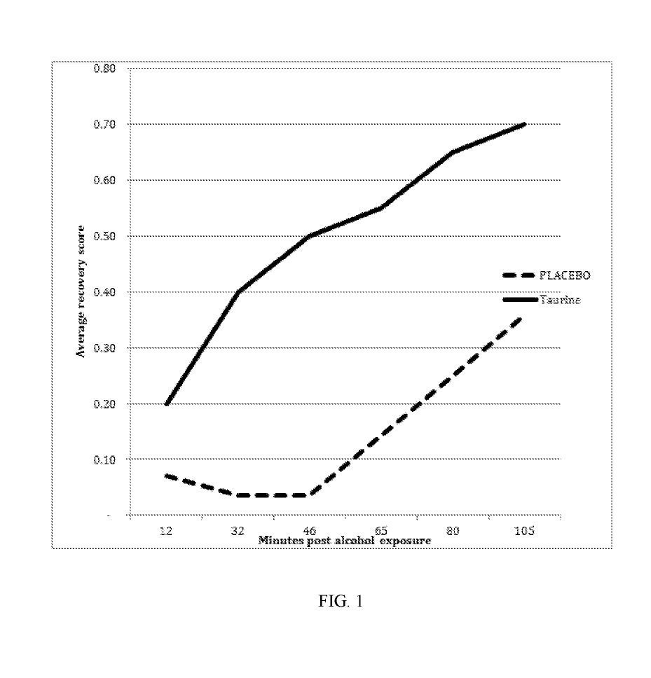

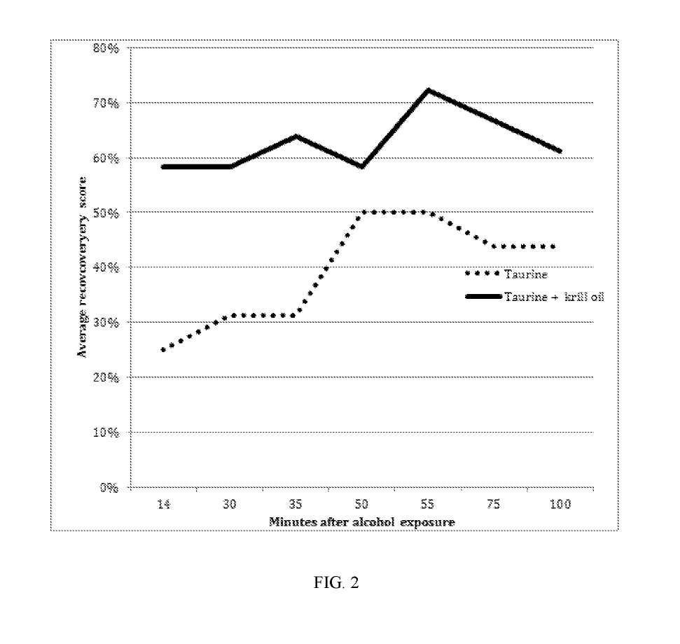

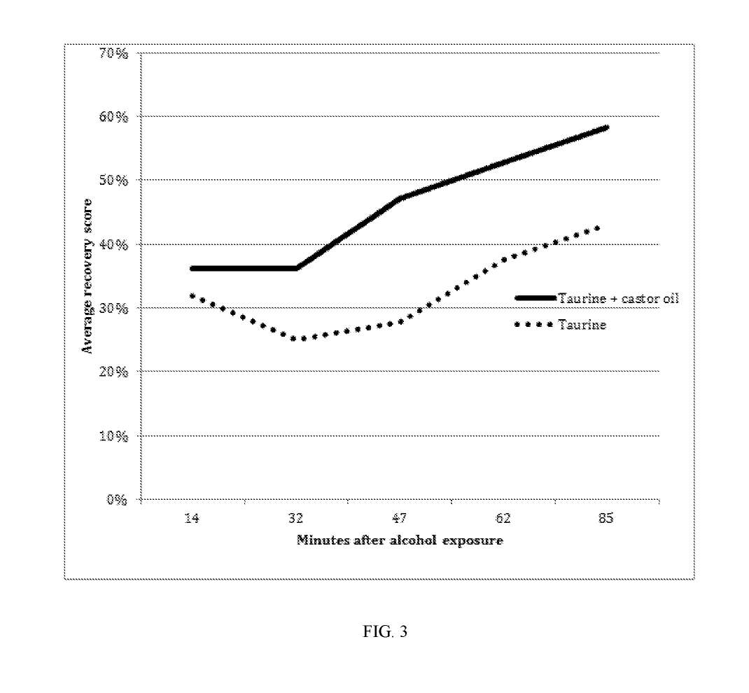

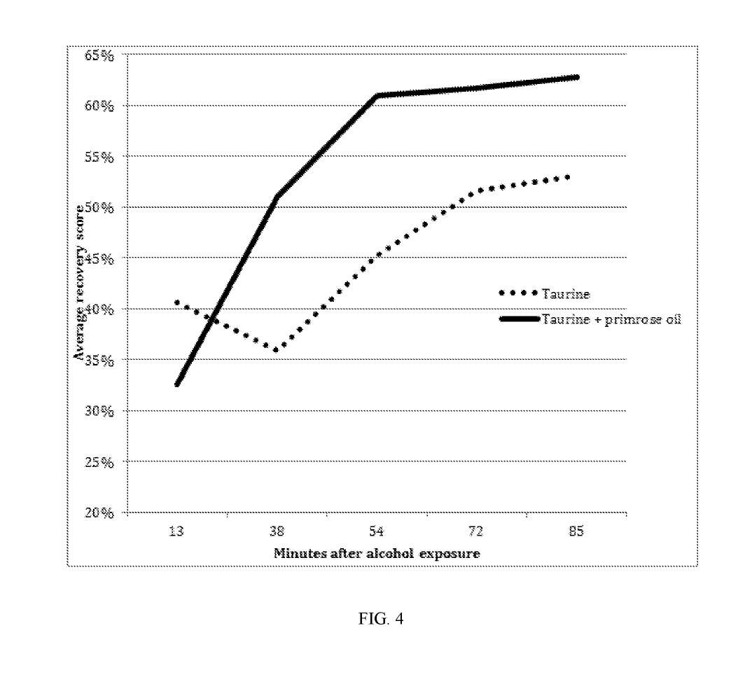

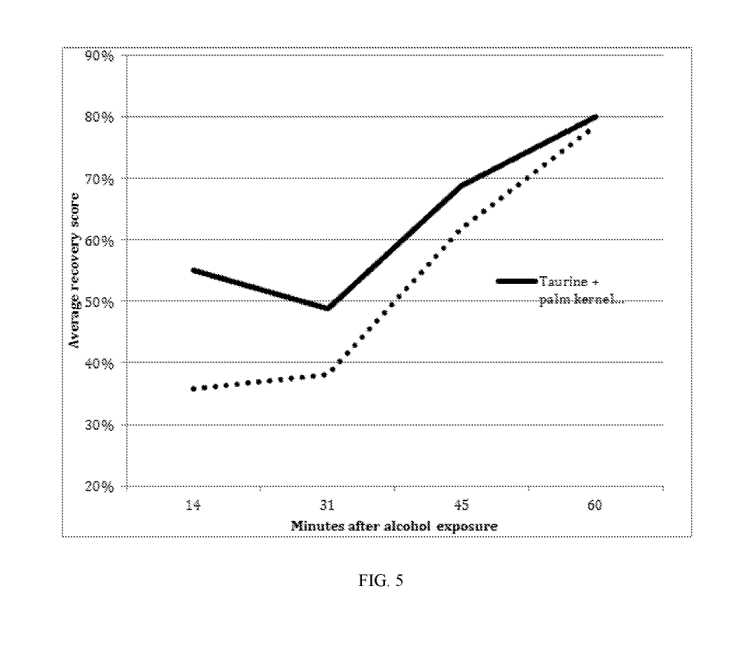

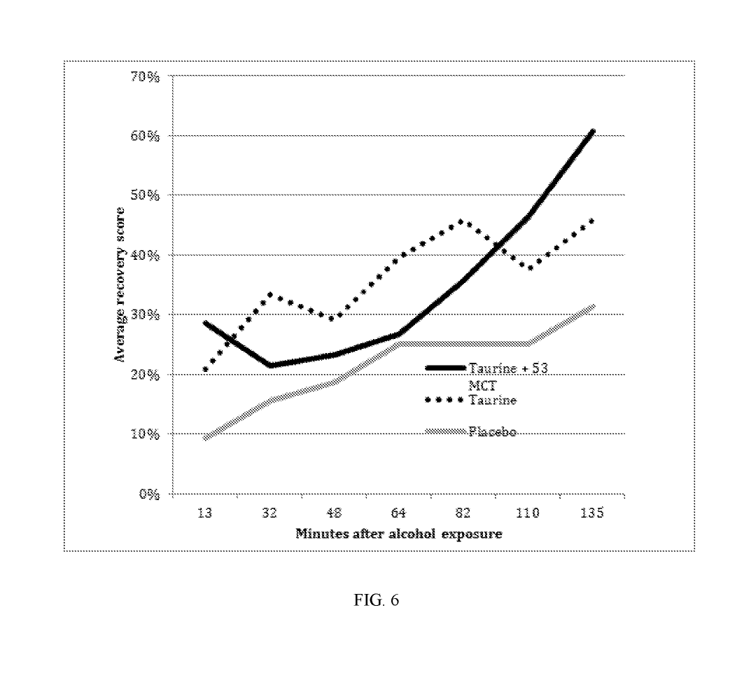

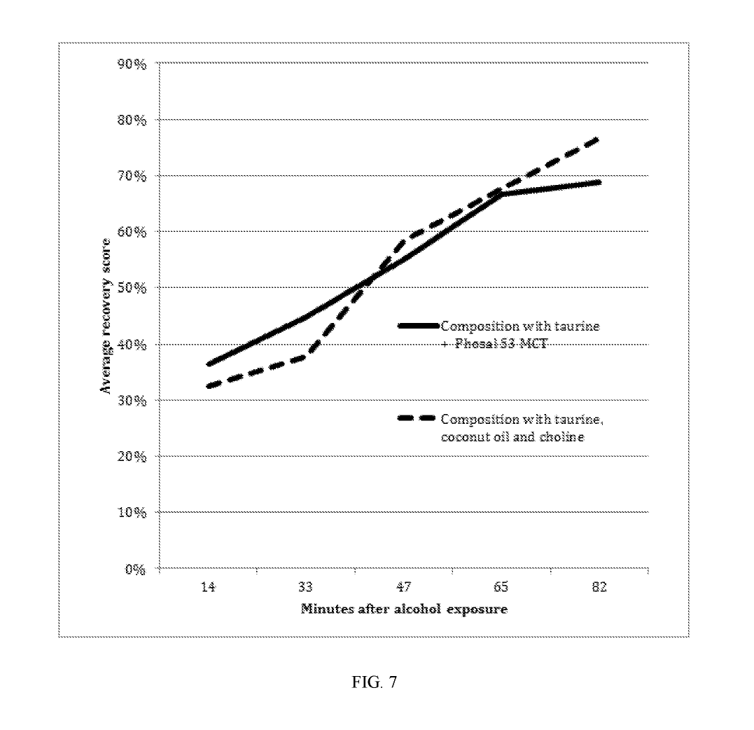

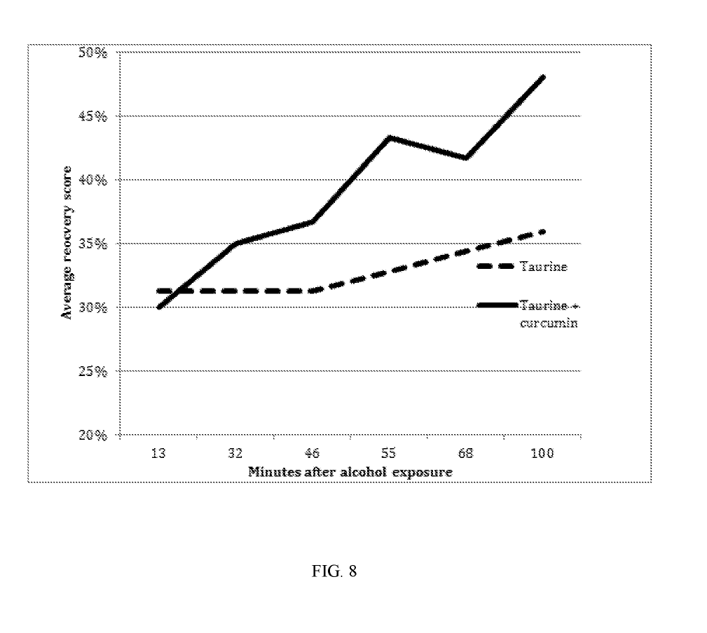

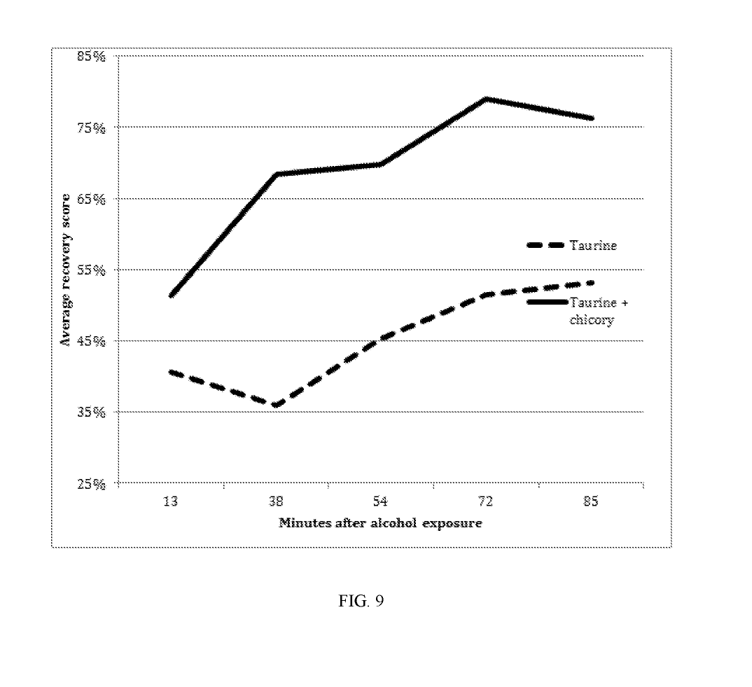

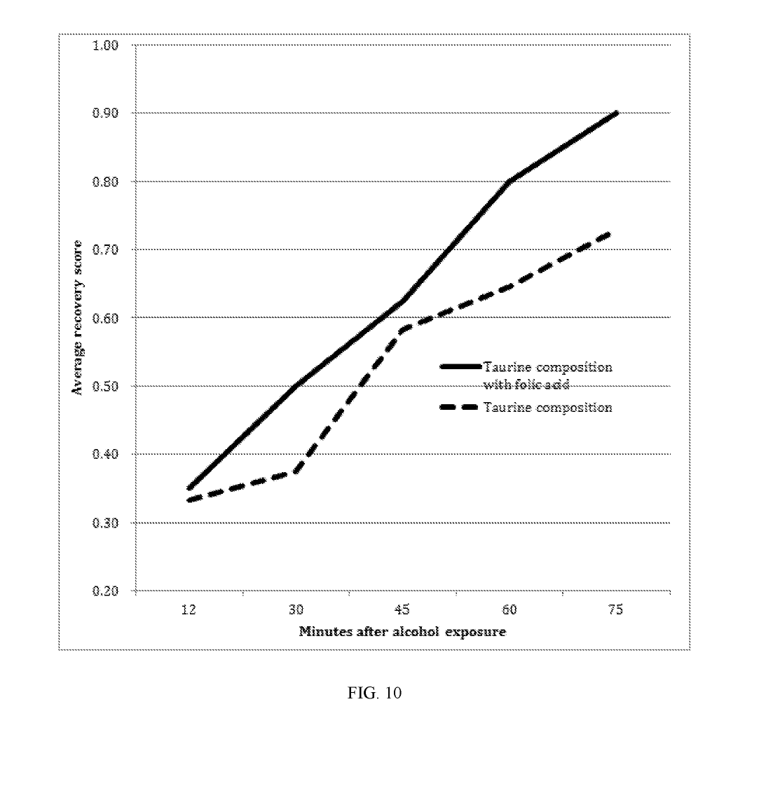

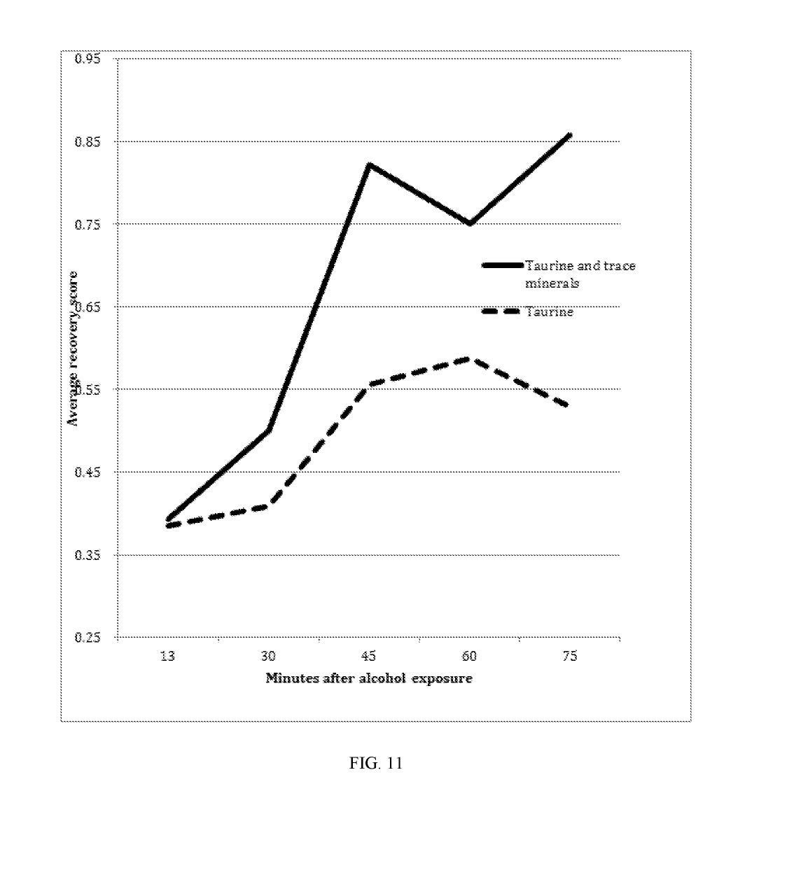

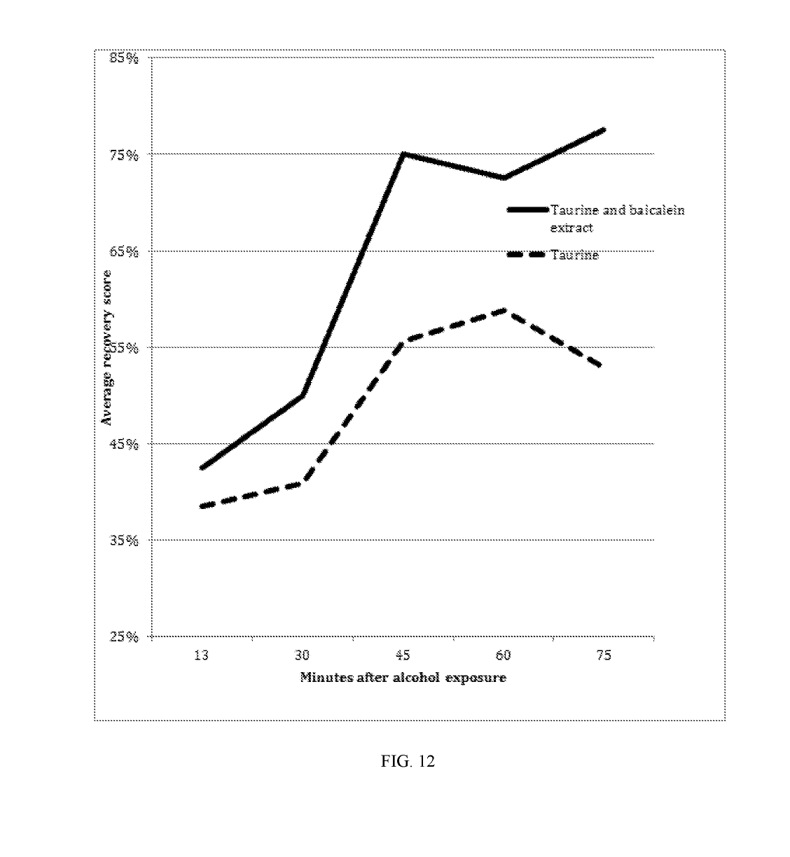

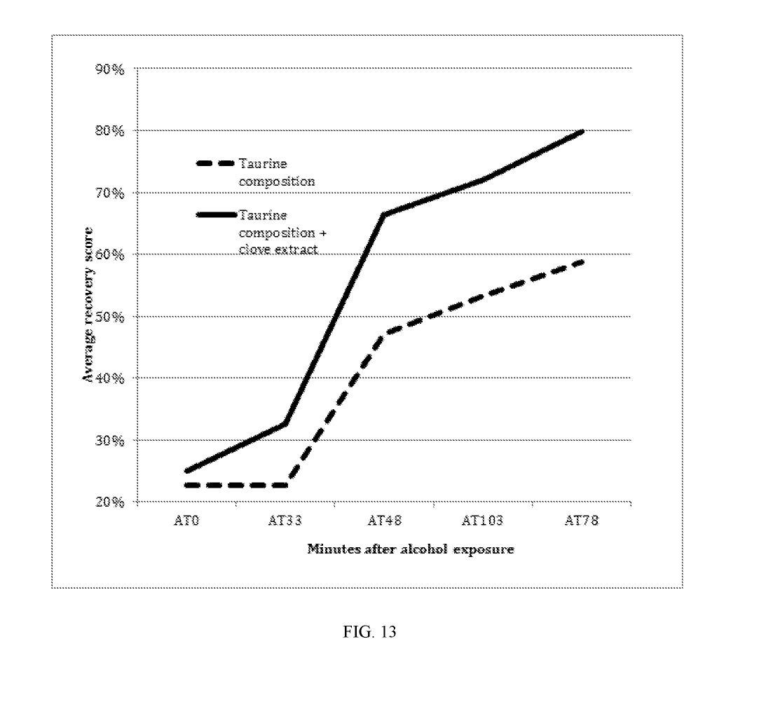

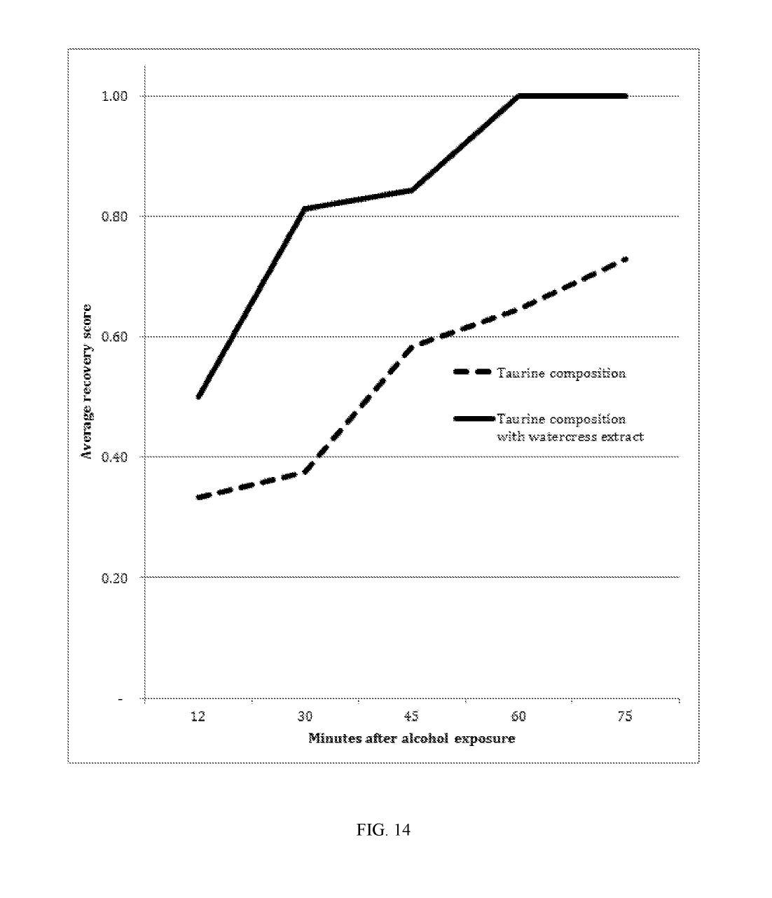

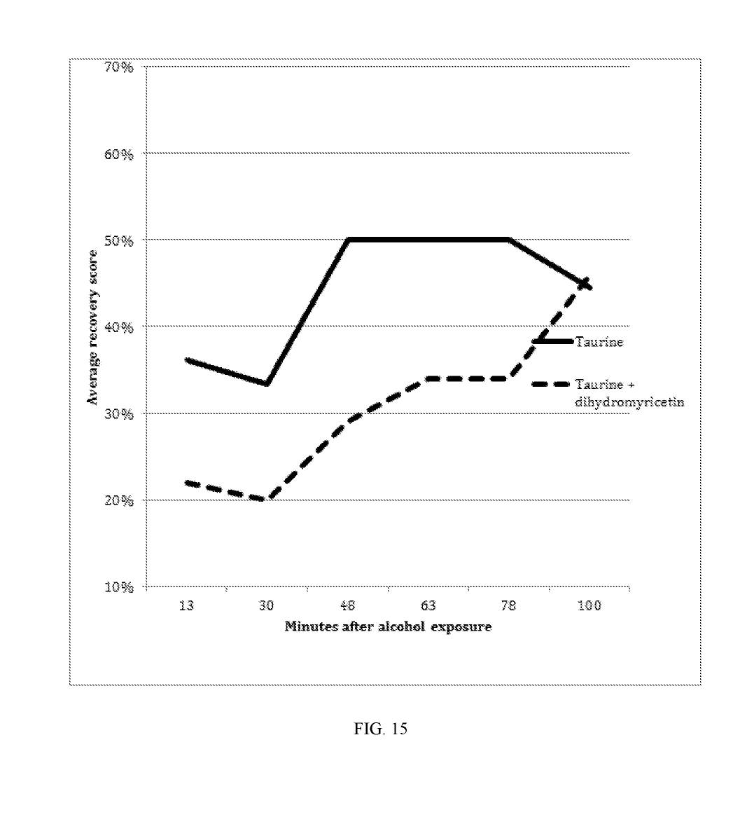

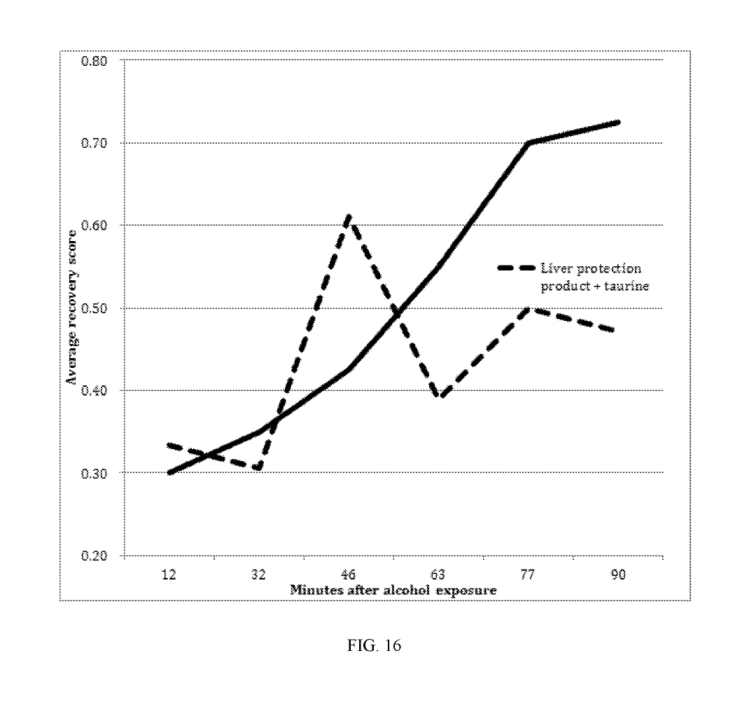

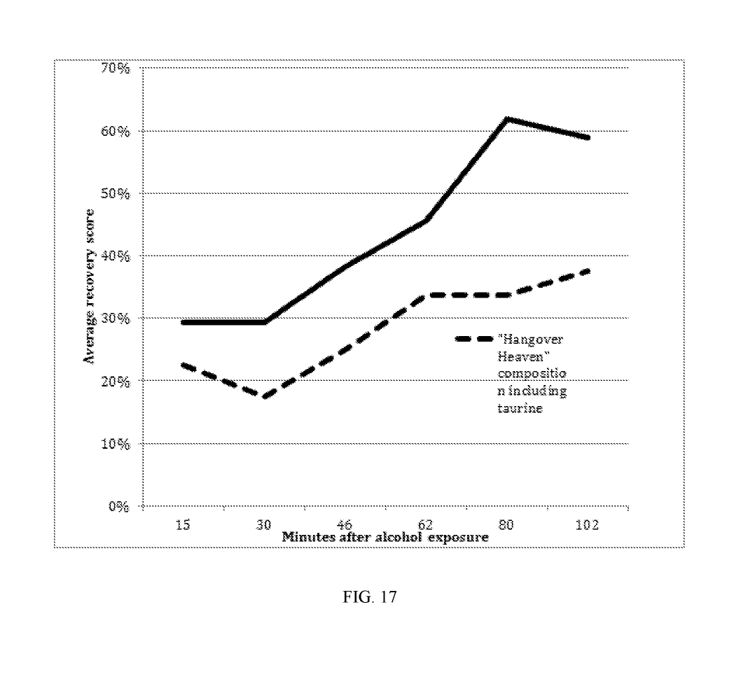

FIG. 1 shows the effects of taurine in an animal model of acute alcohol exposure over time. Based on the results seen in FIG. 1, taurine accelerates recovery from consumption of alcohol in comparison with no taurine added (using a placebo). FIG. 2 shows the effects of Krill oil in combination with taurine in an animal model of acute alcohol exposure over time relative to taurine supplementation alone. Based on the results seen in FIG. 2, using Krill oil in combination with taurine accelerates recovery from consumption of alcohol better than taurine supplementation alone. FIG. 3 shows the effects of Castor oil in combination with taurine in an animal model of acute alcohol exposure over time relative to taurine supplementation alone. Based on the results seen in FIG. 3, using Castor oil in combination with taurine accelerates recovery from consumption of alcohol better than taurine supplementation alone. FIG. 4 shows the effects of Primrose oil in combination with taurine in an animal model of acute alcohol exposure over time relative to taurine supplementation alone. Based on the results seen in FIG. 4, using Primrose oil in combination with taurine accelerates recovery from consumption of alcohol better than taurine supplementation alone. FIG. 5 shows the effects of Palm kernel oil in combination with taurine in an animal model of acute alcohol exposure over time relative to taurine supplementation alone. Based on the results seen in FIG. 5, using Palm kernel oil in combination with taurine accelerates recovery from consumption of alcohol better than taurine supplementation alone. FIG. 6 shows the effects of Phosal® 53 MCT (soy-based lecithin oil plus medium chain triglycerides) in combination with taurine in an animal model of acute alcohol exposure over time relative to taurine supplementation alone or no added taurine (using a placebo). Based on the results seen in FIG. 6, using Phosal® 53 MCT in combination with taurine accelerates recovery from consumption of alcohol better than taurine supplementation alone. FIG. 7 shows the effects of using a composition composed of coconut oil, choline and taurine in an animal model of acute alcohol exposure over time relative to a composition with Phosal® 53 MCT (soy-based lecithin oil plus medium chain triglycerides) and taurine. Based on the results seen in FIG. 7, using a composition of coconut oil and choline in addition to taurine has equivalent efficacy to using a composition with Phosal® 53 MCT and taurine. FIG. 8 shows the effects of Curcumin in combination with taurine in an animal model of acute alcohol exposure over time relative to taurine supplementation alone. Based on the results seen in FIG. 8, using Curcumin in combination with taurine accelerates recovery from consumption of alcohol better than taurine supplementation alone. FIG. 9 shows the effects of Chicory extract in combination with taurine in an animal model of acute alcohol exposure over time relative to taurine supplementation alone. Based on the results seen in FIG. 9, using chicory extract in combination with taurine accelerates recovery from consumption of alcohol better than taurine supplementation alone. FIG. 10 shows the effects of folic acid in combination with taurine in an animal model of acute alcohol exposure over time relative to taurine supplementation alone. Based on the results seen in FIG. 10, using folic acid in combination with taurine accelerates recovery from consumption of alcohol better than taurine supplementation alone. FIG. 11 shows the effects of adding trace minerals to a composition including taurine in an animal model of acute alcohol exposure over time relative to taurine supplementation alone. Based on the results seen in FIG. 11, adding trace minerals in combination with taurine accelerates recovery from consumption of alcohol better than taurine supplementation alone. FIG. 12 shows the effects of Baicalein extract in combination with taurine in an animal model of acute alcohol exposure over time relative to taurine supplementation alone. Based on the results seen in FIG. 12, adding trace minerals in combination with taurine accelerates recovery from consumption of alcohol better than taurine supplementation alone. FIG. 13 shows the effects of adding clove extract to a composition including taurine in an animal model of acute alcohol exposure over time relative to taurine supplementation alone. Based on the results seen in FIG. 13, adding clove extracts in combination with taurine accelerates recovery from consumption of alcohol better than taurine supplementation alone. FIG. 14 shows the effects of adding watercress extract to a composition including taurine in an animal model of alcohol exposure over time compared to using the same composition without watercress extract. Based on the results seen in FIG. 14, using watercress extracts in combination with taurine accelerates recovery from consumption of alcohol better than taurine supplementation alone. FIG. 15 shows the effects of adding dihydromyricetin extract to taurine supplementation in an animal model of alcohol exposure over time compared to taurine supplementation alone. Based on the results seen in FIG. 15, using dihydromyricetin extracts in combination with taurine does not accelerate recovery from consumption of alcohol better than taurine supplementation alone. FIG. 16 shows the effects of adding taurine to a commercial liver protection product in an animal model of alcohol exposure over time compared to using the commercial liver protection product without taurine. Based on the results seen in FIG. 16, adding taurine to a commercial liver protection product interrupts recovery from exposure to alcohol compared to using a commercial liver protection product alone without taurine. This example demonstrates that adding taurine to otherwise efficacious ingredients can disrupt the efficacy of the other ingredients without demonstrating additive effects. In other words, not all compositions that contain taurine are equally effective and in some cases adding taurine decreases efficacy. FIG. 17 shows a commercial hangover product (“Hangover Heaven”) which contains taurine as one of many ingredients in an animal model of alcohol exposure over time compared to an invention composition including taurine. Based on the results seen in FIG. 17, it is clear that some compositions containing taurine as exemplified by the Hangover Heaven composition are not as effective at accelerating recovery from consumption of alcohol as using an invention composition including taurine.

Human Testing of Alcohol Exposure

In the following examples, preferred embodiments of the invention in humans were performed to confirm the animal efficacy data. Volunteers were asked to follow a testing protocol that required them to consume a given amount of alcohol over a given period of time with or without food as they preferred. They would measure their blood alcohol level at various intervals after consuming alcohol using a professional breathalyzer device. Volunteers would conduct the protocol twice: once in the context of consuming the combination of natural products and once without. The efficacy of a preferred embodiment of the invention in the context of consumption of beer, wine, spirits, and mixed drinks by men and women both fed and fasted were tested.

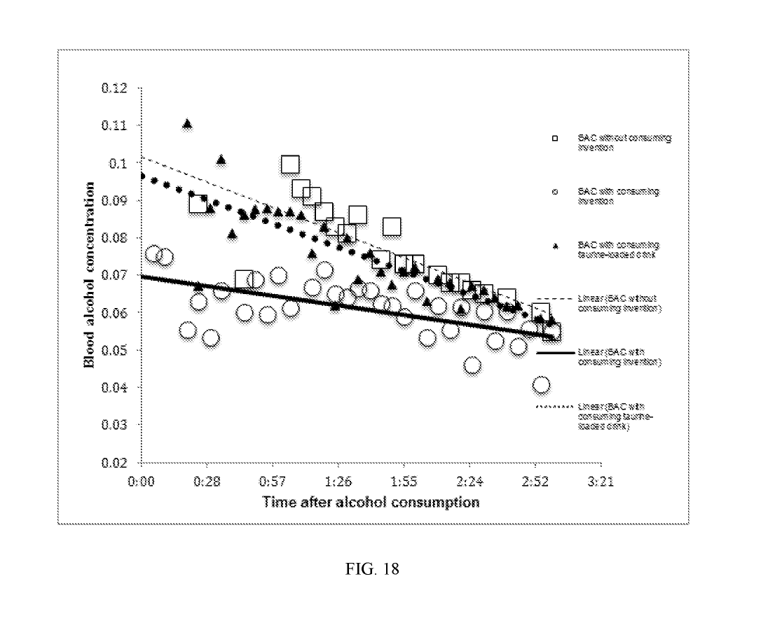

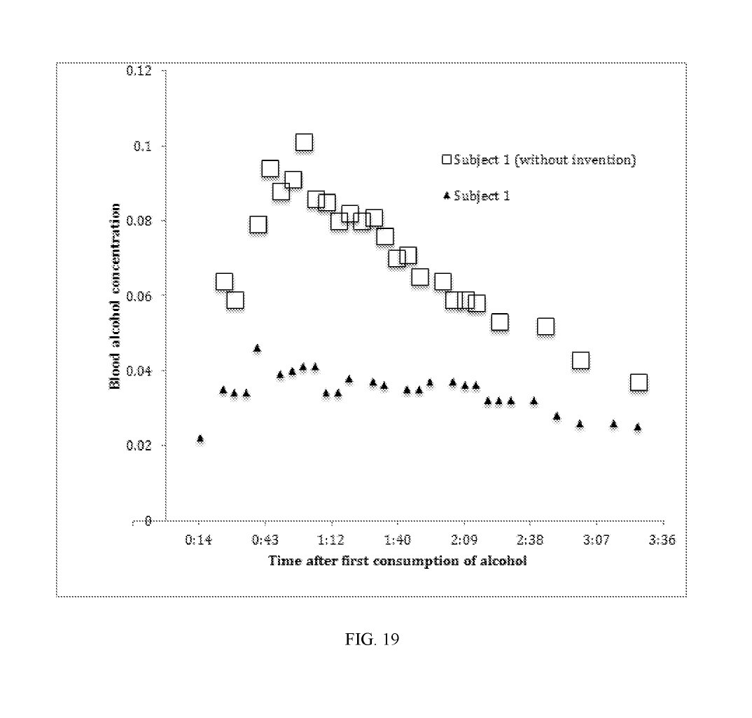

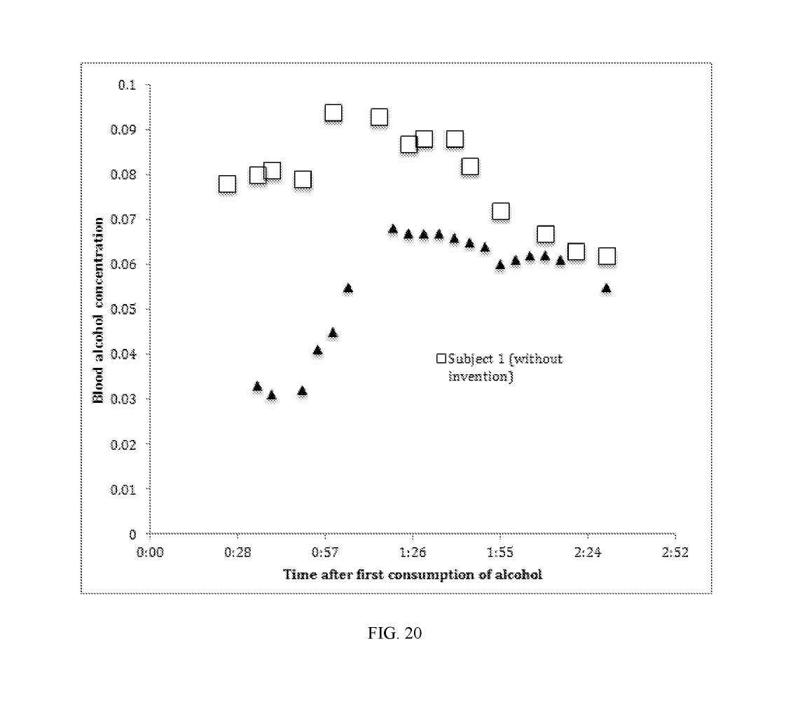

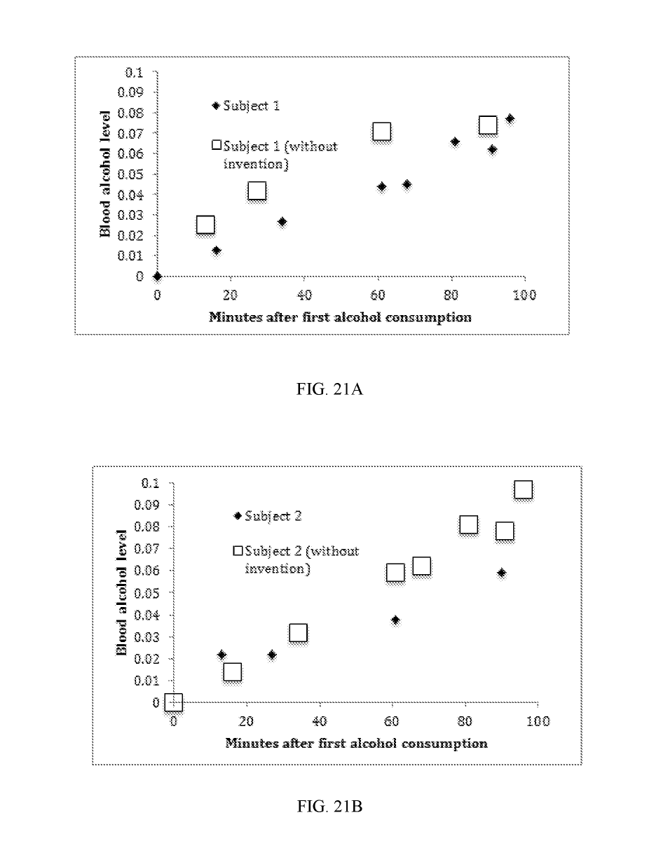

FIG. 18 shows the effects of a composition according to an embodiment of the invention on blood alcohol levels (blood alcohol concentration) over time in a person from a “binge drinking” experiment. In this example, a volunteer consumed 200 ml of vodka (40% alcohol by volume) over a span of thirty minutes in conjunction with either drinking a dose of a composition according to the invention in liquid form or not. All drinking was done after consuming meals of comparable size and composition. Blood alcohol levels were recorded with a professional breathalyzer device. Based on the results seen in FIG. 18, the blood alcohol concentration over time after alcohol consumption of the person consuming a composition according to the invention (represented by circles) decreases the fastest compared to the same person not consuming a composition according to the invention (represented by squares) or compared to the same person consuming a composition containing taurine alone (represented by triangles). Consequently, the person consuming a composition according to the invention recovers fastest from the toxicity of alcohol. FIG. 19 shows the effects of a composition according to an embodiment of the invention on blood alcohol levels over time in a person from a “binge drinking” experiment on an empty stomach. The same volunteer as in the previous example (FIG. 18) consumed 200 ml of vodka (40% alcohol by volume) over a span of thirty minutes in conjunction with either drinking a dose of a composition according to the invention in liquid form or not. The volunteer consumed no food for four hours prior to drinking and testing. Blood alcohol levels were recorded with a professional breathalyzer device. Based on the results seen in FIG. 19, the blood alcohol concentration over time after alcohol consumption of the person (Subject 1) consuming a composition according to the invention (represented by triangles) decreases the fastest compared to the same person not consuming a composition according to the invention (represented by squares). Consequently, the person consuming a composition according to the invention recovers fastest from the toxicity of alcohol. FIG. 20 shows the effects of a composition according to an embodiment of the invention on blood alcohol levels over time in a person from a “binge drinking” experiment on an empty stomach. The same volunteer as in the previous example (FIG. 19) consumed 150 ml of vodka (40% alcohol by volume) over a span of thirty minutes in conjunction with either drinking a dose of a composition according to the invention in liquid form or not. The volunteer consumed no food for four hours prior to drinking and testing. Blood alcohol levels were recorded with a professional breathalyzer device. Based on the results seen in FIG. 20, the blood alcohol concentration over time after alcohol consumption of the person (Subject 1) consuming a composition according to the invention (represented by triangles) decreases the fastest compared to the same person not consuming a composition according to the invention (represented by squares). Consequently, the person consuming a composition according to the invention recovers fastest from the toxicity of alcohol. FIGS. 21A and 21B each shows the effects of a composition according to an embodiment of the invention on blood alcohol levels over time in a person from a “cocktails” experiment. Two volunteers (female as Subject 1 (FIG. 21A), and male as Subject 2 (FIG. 21B)) each consumed four 58 ml of bourbon (40% alcohol by volume) over a span of one hour and fifteen minutes in conjunction with either drinking a dose of a composition of the present invention according to the invention in liquid form or not. On both occasions volunteers consumed meals of comparable size and composition. Blood alcohol levels were recorded with a professional breathalyzer device. Based on the results seen in FIGS. 21A and 21B, the blood alcohol concentration over time after alcohol consumption of the persons (Subject 1 and Subject 2) consuming a composition according to the invention was substantially lower than when the persons did consume a composition according to the invention.

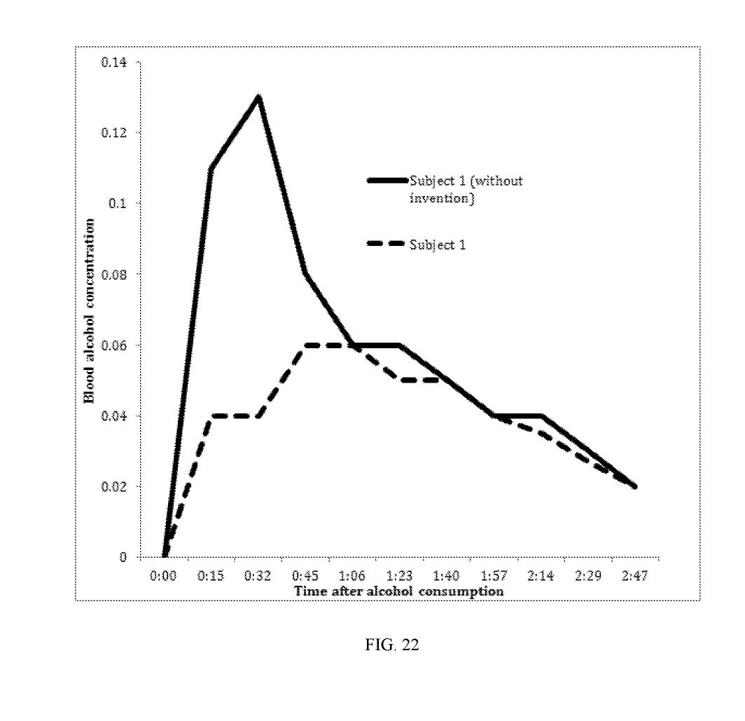

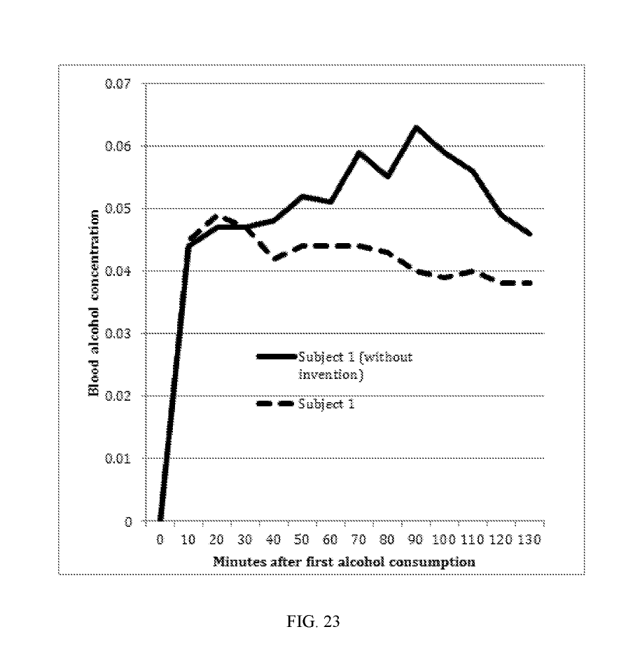

FIG. 22 shows the effects of a composition according to an embodiment of the invention on blood alcohol levels over time in humans from a “wine drinking” experiment. In this example, a female volunteer consumed 13 ounces of red wine (13% alcohol by volume) over a span of thirty minutes in conjunction with either drinking a dose of a composition according to the invention in liquid form or not. On both occasions the volunteer consumed meals of comparable size and composition. Blood alcohol levels were recorded with a professional breathalyzer device. Based on the results seen in FIG. 22, the blood alcohol concentration of the female subject after taking a composition according to the invention was reduced considerably over time as captured in hours and fractions thereof. FIG. 23 shows the effects of a composition according to an embodiment of the invention on blood alcohol levels over time in humans from a “beer drinking” experiment. In this example, a male volunteer consumed two 12 ounce bottles of beer (8% alcohol by volume) over a span of ninety minutes in conjunction with either drinking a dose of a composition of the invention in liquid form or not. On both occasions the volunteer consumed meals of comparable size and composition. Blood alcohol levels were recorded with a professional breathalyzer device. Based on the results seen in FIG. 23, the blood alcohol concentration of the male subject after taking a composition according to the invention was reduced considerably over time with the effect lasting well past 2 hours.

Compositions may be formulated as desired and a number of inactive ingredients may be added, for example, but not limited to provide longer shelf life, to make the formulation more palatable or presentable, or to decrease manufacturing costs.

In certain aspects of the present invention, a composition is for oral administration in a variety of forms, including, without limitation, liquid tincture, beverage, food product such as, but not limited to a health bar or a pudding, confectionaries, lozenge, tablet, caplet, capsule or the like. A composition is preferably added to, for example, drinks that are light and easy to drink before alcohol consumption, confectionaries such as chewable tablets to go with alcohol. Pharmaceutically acceptable excipients, solvents, carriers or diluents are well known to the skilled person and may be incorporated as desired. In solid dosage forms for oral administration an active compound(s) may be admixed with an inert excipient such as, but not limited to sodium citrate or dicalcium phosphate; a filler, such as but not limited to starch, lactose, sucrose, glucose, mannitol or silicic acid; a binder, humectant, disintegrating agent, solution retarder, absorption accelerator, wetting agent, absorbent, or any combination thereof as desired.

The daily dosage of compounds may be administered in the form of one or more unit dosage forms, such as a capsule, caplet, tablet or the like. The formulation of an individual dosage unit can be based on the amount of active ingredient(s) that are required to be present in each tablet to total the amount of active ingredient(s) required daily. Extra amounts may be incorporated into the formulation in order to compensate for degradation of active ingredients over time.

The present invention also provides a method of treating a negative effect of alcohol consumption in a person. The method comprises administering to such a person a composition in one or more unit dosage form comprising taurine, a lipid and a natural or synthetic compound. A composition may be administered orally prior to alcohol consumption, during alcohol consumption, after alcohol consumption or any combination thereof. A unit dose is defined as comprising between about 0.5 and about 6 grams of taurine and between about 2 and about 40 grams of a lipid. In another embodiment, a unit dose can include between about 0.5 and about 6 grams of taurine, between about 2 and about 40 grams of a lipid, and between about 1 and about 40 grams of a natural or synthetic compound. In yet another embodiment, a unit dose can include between about 0.5 and about 6 grams of taurine, and between about 1 and about 40 grams of a natural or synthetic compound. In a preferred embodiment, a unit dose can include between about 2 to about 6 grams of taurine, between about 5 to about 30 grams of a lipid, and between about 3 to about 20 grams of a natural or synthetic compound. In a preferred embodiment, one unit dose of a composition is administered per drinking session or per day to a person. In an alternate embodiment, a first dose is administered daily independent of alcohol consumption, and a second dose is administered proximally to alcohol consumption. In a preferred embodiment, a unit dose is administered prior to alcohol consumption and more preferably at least 15 minutes prior to alcohol consumption.

Other objects, advantages and novel features of the present invention are apparent from the foregoing detailed description of the one or more preferred embodiments, examples and aspects. It should be recognized that the one or more examples in the disclosure are non-limiting examples and that the present invention is intended to encompass variations and equivalents of these examples.

What is claimed is:

1. A composition for reducing blood alcohol concentration (BAC) in a person after alcohol consumption, a composition comprising:
taurine present in an amount from about 0.5 to about 6 grams; and
a lipid present in an amount from about 2 to about 40 grams.

2. A composition according to claim 1, wherein the lipid is selected from the group consisting of fats, waxes, sterols, fat-soluble vitamins, monoglycerides, diglycerides, triglycerides, and phospholipids.

3. A composition according to claim 2, wherein the lipid is derived from an oil or oils of plant or animal origin.

4. A composition according to claim 3, wherein the lipid is krill oil, Phosal® 53 MCT, castor oil, primrose oil, palm kernel oil, or coconut oil.

5. A composition according to claim 1, further comprises a natural or synthetic compound present in an amount from about 1 to about 40 grams.

6. A composition according to claim 5, wherein the natural or synthetic compound is selected from herbal extracts and dietary supplements.

7. A composition according to claim 6, wherein the herbal extracts and dietary supplements are selected from the group consisting of curcumin, chicory extract, baicalein extract, clove extract, watercress extract, choline, and folic acid.

8. A composition according to claim 7, wherein the choline is choline chloride, choline bitartrate, citicoline (CDP-choline), L-alpha-glycerophosphocholine (Alpha-GPC), lecithin, or phosphatidylcholine (PC).

9. A composition according to claim 1, wherein a composition is formulated into an oral dosage form.

10. A composition according to claim 9, wherein the oral dosage form is selected from the group consisting of: liquid tincture, beverage, food product, lozenge, tablet, caplet, and capsule.

11. A composition for reducing blood alcohol concentration (BAC) in a person after alcohol consumption, a composition comprising:
taurine present in an amount from about 0.5 to about 6 grams; and
a natural or synthetic compound present in an amount from about 1 to about 40 grams.

12. A method of treating a negative effect of alcohol consumption in a person in need of such treatment, the method comprising:
orally administering to said person a composition comprising taurine present in an amount from about 0.5 to about 6 grams and a lipid present in an amount from about 2 to about 40 grams.

13. The method according to claim 12, wherein a composition further comprises a natural or synthetic compound present in an amount from about 1 to about 40 grams.

14. The method according to claim 12, wherein the lipid is selected from an oil or oils of plant or animal origin.

15. The method according to claim 13, wherein the natural or synthetic compound are selected from herbal extracts and dietary supplements.

16. The method according to claim 12, wherein the composition is administered to the person prior to consuming alcohol.
